(19) United States
(12) Patent Application Publication
(10) Pub. No.: US 2007/0043381 A1
(43) Pub. Date: Feb. 22, 2007

(54) Medical device deployment instrument

(75) Inventors: Joseph G. Furst, Lyndhurst, OH (US); Ravish Sachar, Raleigh, NC (US); Perry J. Katzenstein, Chesterland, OH (US)

Correspondence Address: FAY, SHARPE, FAGAN, MINNICH & MCKEE, LLP, 1100 SUPERIOR AVENUE, SEVENTH FLOOR, CLEVELAND, OH, 44114, US

(73) Assignee: Icon Medical Corp.

(21) Appl. No.: 11/507,136

(22) Filed: Aug. 18, 2006

Related U.S. Application Data
(60) Provisional application No. 60/709,575, filed on Aug. 19, 2005.

(51) Int. Cl. A61F 11/00

(52) U.S. Cl. 606/108; 623/1.11

(57) ABSTRACT

A deployment device for deploying a medical device in-a body passageway. The deployment device designed to deploy at least a portion of a proximal end of the medical device before partially or fully deploying a distal end of the medical device.

The present invention claims priority on U.S. Provisional Application Ser. No. 60/709,575 filed Aug. 19, 2005, which is incorporated herein by reference

### FIELD OF THE INVENTION

The present invention is directed to medical devices, and more particularly to instruments and procedures for deploying medical devices in a body passageway.

### BACKGROUND OF THE INVENTION

In the field of interventional cardiology, many devices are utilized to cure arterial disease. Arterial disease can be described as a narrowing of a vein or artery in a mammalian host. Arterial disease can be cured or treated with a variety of methods including surgery or less invasive methods such as procedures defined in the field of interventional cardiology. Interventional cardiology utilizes devices including, but not limited to, atherectomy, angioplasty balloons, rotoblator, extraction devices, distal protection devices, stents, and most notably drug eluting stents and ostial stents. These stents have been manufactured from a multitude of metals including stainless steel, cobalt chromium, magnesium, and nickel-titanium, commonly referred to as nitinol. Nitinol is a self-expanding memory metal.

Stents, although effective in the treatment of coronary artery disease, have physical limitations. These limitations are not due solely to the metal or device itself, but can also be due to the procedure by which the device is delivered. Some stents are designed to be self-expanding, thus the stent must be physically retained in a non-fully expanded shape until the physician has manipulated the delivery catheter to the sight of the vessel blockage or occlusion. Once the stent is situated in the correct position, the physician mechanically releases the stent by removing a sheath cover and thereby deploying the stent. The stent deployment process is designed to deliver the stent from the furthest (distal) end first, then followed by the closest (proximal) end of the stent to the operator. The stent, although deployed, has a tendency to “jump” past the blockage or occlusion into the artery because of the inherent properties of the self expanding metal. As such, the stent may not be in the proper or desired position after deployment. Another problem encountered with self expanding stents is that the stent is difficult to properly position, especially at an ostium of a blood vessel. Various types of stents have been developed for use at an ostium of a blood vessel. Examples of such stents are disclosed in U.S. Pat. Nos. 4,994,071; 5,456,712; 5,466,242; 5,607,144 and 6,293,964, all of which are incorporated herein by reference. When such stents are inserted into a blood vessel, the distal end of the stent is first deployed to maintain the stent within the blood vessel. Thereafter, the stent cover or shealth is continually removed until the proximal end of the stent is uncovered. The distal end of the stent, when properly positioned, results in the proximal end of the stent at least partially conforming to the ostium of the blood vessel. However, when the distal end is not properly positioned, the proximal end of the stent either sticks to far out from the ostium or does not properly expand to properly conform to the ostium of the blood vessel.

In view of the current state of the art for deploying medical devices such as, but not limited to, stents in a body passageway, there is a need for a device and method that can be used to accurately deploy a medical device in a body passageway.

### SUMMARY OF THE INVENTION

The present invention is directed to a novel deployment device and methodology for the deployment of a medical device into a body passageway. As defined herein, the term “body passageway” is defined to be any passageway or cavity in a living organism (e.g., bile duct, bronchiole tubes, nasal cavity, blood vessels, heart, esophagus, trachea, stomach, fallopian tube, uterus, ureter, urethra, the intestines, lymphatic vessels, nasal passageways, eustachian tube, acoustic meatus, etc.). The novel deployment device is particularly directed to the deployment of a medical device such as, but not limited to, a stent into a body passageway such as, but not limited to, a blood vessel, and thus the invention will be described with particular reference to a stent and blood vessels; however, it will be appreciated that the invention has much broader applications, and thus can be used to deploy other types of medical devices into blood vessels and/or other types of body passageways. The deployment device is designed to deploy at least a portion of a proximal end of a medical device prior to partially or fully deploying a portion of a distal end of the medical device. Such a deployment method has heretofore not been achieved since prior art deployment devices deploy the distal end of the medical device prior to deploying the proximal end of the medical device. The deployment method in accordance with the present invention has been found to be advantageous for deploying medical devices such as, but not limited to, a stent in a body passageway such as, but not limited to, a blood vessel.

In one non-limiting aspect of the present invention, the deployment device can include, but is not limited to, a first tube, a second tube, and a third tube. The first tube (e.g., medical device deployment tube) has an inner diameter configured to receive an associated guide wire or other type of guiding device (e.g., over-the-wire delivery system, monorail type delivery system, etc.). Common guide wire diameters, when used, include, but are not limited to, 0.014 inch, 0.018 inch and 0.035 inch; however, other guide wire diameters can be used in association with the present invention. The second tube (e.g., medical device mounting tube) has an inner diameter configured to receive the first tube and has an outer diameter configured to be received by an associated stent or other type of medial device. In one non-limiting arrangement, a stent can be mounted on the outer diameter of the second tube. In another non-limiting arrangement, a balloon and a stent can be mounted on the outer diameter of the second tube. In one or both of the non-limiting arrangements previously mentioned, an adhesive can be used to at least partially secure the stent to the second tube and/or be used to at least partially secure the balloon to the stent and/or second tube; however, this is not required. The third tube (e.g., medical device cover) has an inner diameter that receives the stent or other type of medical device. The first tube contacts and/or engages the third tube. Such contact and/or engagement is generally at or adjacent a distal end of the first tube; however, this is not required. As can be appreciated, the first tube can be connected to the third tube; however, this is not required. The third tube is designed to deploy a stent or other type of medical device by moving the third tube in an axial direction with respect to the second tube. As can be appreciated, the tubes of the novel deployment device can be made of the same of different material. Generally, the material used to form the novel deployment device includes polymer materials and/or metal materials; however, this is not required. The one or more tubes can be solid tubes, braided tubes, etc. The first and second tubes are generally formed of a flexible material to facilitate in the positioning of these tubes in a body passageway; however, this is not required. The third tube can be made of a rigid and/or flexible material. The one or more tubes of the novel deployment device generally have a circular cross-sectional area; however, it can be appreciated that other shapes can be used for one or more of the tubes (e.g., oval, polygonal, etc.). In one non-limiting embodiment, the third tube is designed to be moved forwardly relative to the second tube so that at least a portion of a proximal end of the medical device is deployed (e.g., uncovered, etc.) prior to deploying a portion of a distal end of the medical device. In an additional and/or alternative non-limiting embodiment of the invention, at least a portion of the outer diameter or cross-sectional area of the third tube is smaller than the inner diameter or cross-sectional area of the deployed medical device so that the third tube can be at least partially drawn through the medical device after it has been at least partially deployed in a body passageway. In one non-limiting design, the outer diameter or cross-sectional area of the third tube is about 10-500% less than the inner diameter or cross-sectional area of the deployed medical device. In still yet an additional and/or alternative non-limiting embodiment, the third tube is at least partially secured to the first tube so that the first tube can be used to at least partially draw the third tube at least partially through the deployed medical device. In one non-limiting configuration, the first tube is at least partially secured to the third tube at and/or near the front end of the first tube and at and/or near the front end of the third tube. In an additional and/or alternative non-limiting configuration, the first tube and third tubes are at least partially connected together by an adhesive, melting, stitching, and/or one or more other types of connection arrangements. In a further and/or alternative non-limiting embodiment, one or more of the three tubes can include one or more openings to allow fluid flow into and/or out of the one or more tubes. These one or more openings are generally located at and/or near the front portion of the one or more tubes; however, this is not required. The one or more openings can be used to 1) facilitate in removing air from the tubes, and/or 2) allowing medicine and/or other types of fluid to be conveyed to a treatment site; however, the one or more openings can be used for other or additional reasons. In a still further and/or alternative non-limiting embodiment, one or more of the three tubes can include one or more markers. The one or more markers can be used to 1) facilitate in informing the physician the location of the medical device and/or one or more components of the novel deployment device in a body passageway, 2) facilitate is positioning the medical device and/or one or more components of the novel deployment device in a body passageway, and/or 3) at least partially retaining the medical device on and/or in position on one or more components of the novel deployment device. As can be appreciated, the one or more markers can have other or additional uses.

In an additional and/or alternative non-limiting aspect of the present invention, one or more of the three tubes can include a tapered end portion and/or a flared end portion. A tapered end portion on one or more of the tubes can be used to facilitate in moving the one or more tubes in a body passageway. The tapered end portion is generally located on the proximal end of one or more tubes; however, this is not required. In one non-limiting embodiment, the third tube includes a tapered end portion on the proximal end of the tube. In an additional and/or alternative non-limiting embodiment, the third tube includes a flared end portion. In one non-limiting aspect of this embodiment, the flared end portion is located on the distal end of the third tube. The flared end portion can be used to facilitate in moving the third tube over a portion of a medical device that has been partially deployed. In one non-limiting aspect of this embodiment, the flared end portion of the third tube is designed to facilitate in moving over a portion of a stent that has been partially deployed. In some instances during the deployment of the stent, it may be determined that the stent is not properly positioned in a body passageway. In such situations, the third tube can be moved back over the exposed portion of the stent so that the stent is compressed to a smaller cross section area to enable the stent to be properly positioned in the body passageway. As such, the stent can be at least partially retrieved back into the deployment device after partial deployment of the stent. When a flared end portion is used on a tube, the flared end portion is generally about 2-45° relative to the longitudinal axis of the tube, typically about 10-30° relative to the longitudinal axis of the tube; however, other angles of flare can be used. The angle of flare can be uniform or non-uniform. When a tapered end portion is used on a tube, the tapered portion is generally about 2-45° relative to the longitudinal axis of the tube, typically about 10-30° relative to the longitudinal axis of the tube; however, other angles of flare can be used. The angle of taper can be uniform or non-uniform. The tapered portion of the tube can be formed of a soft material that minimizes trauma to a body passageway as the one or more tubes are moved in the body passageway; however, this is not required. The soft material is typically formed of a different material and/or has a different thickness from other portions of the tube; however, this is not required.

In an additional and/or alternative non-limiting aspect of the present invention, the deployment device can include a fixture that is designed to inhibit or prevent a medical device from undesirable movement relative to one or more tubes of the deployment device during the deployment of the medical device. Axial movement of the medical device on the deployment device during the deployment of the medical device can result in the improper positioning of the medical device in a body passageway. The fixture in the deployment device is designed to reduce or prevent such axial movement of the medical device during deployment of the medical device. In one non-limiting embodiment of the invention, the fixture is positioned at least partially about the second tube. The fixture can be positioned, connected to, and/or formed on the second tube. In this non-limiting arrangement, at least a portion of the fixture forms a barrier in the space between the outer surface of the second tube and the inner surface of the third tube. This barrier formed by the fixture inhibits or prevents axial movement on the second tube of the medical device in at least one direction when the third tube is moved relative to the second tube. The fixture can be formed of the same or different material than one or more tube of the deployment device. In one non-limiting aspect of this embodiment, the fixture is secured to or formed in a portion of the outer surface of the second tube. When the fixture is secured to the second tube, the means for securing can include, but is not limited to, melting, adhesive, mechanical connector, etc. In an additional and/or alternative aspect of this embodiment, the fixture is positioned at or near the proximal end of the second tube. In yet an additional and/or alternative aspect of this embodiment, the fixture has a height that is at least about 20% of the maximum space between the outer surface of the second tube and the inner surface of the third tube when the second tube is positioned in the third tube. In still yet an additional and/or alternative aspect of this embodiment, the fixture has a height that is about 25-100% of the maximum space between the outer surface of the second tube and the inner surface of the third tube when the second tube is positioned in the third tube.

In still an additional and/or alternative non-limiting aspect of the present invention, one or more tubes can include a coating material to facilitate in the movement of the one or more tubes in a body passageway and/or movement of the tubes relative to one another. The coating material can be bio-inert and/or biodegradable; however, this is not required. The coating material can be permanently or releaseably applied to one or more surfaces of one or more tubes. As can be appreciated, a coating material can also be applied to the guide wire or the like to facilitate in the movement of a tube over the guide wire; however, this is not required.

In still an additional and/or alternative non-limiting aspect of the present invention, the deployment device can include at least one marker material to identify the location of 1) one or more tubes relative to one another, 2) the proximal and/or distal end of one or more tubes, 3) the location of a fixture on one or more tubes, and/or 4) the location of one or more portions of the medical device on the deployment device. As can be appreciated, the one or more markers on the deployment device can have other or additional functions. In one non-limiting embodiment of the invention, the deployment device includes at least one marker to identify the distal end of a medical device on the deployment device. In an additional and/or alternative non-limiting embodiment of the invention, the deployment device includes at least one marker to identify the proximal end of a medical device on the deployment device. In still an additional and/or alternative non-limiting embodiment of the invention, the deployment device includes at least one marker to identify the proximal end of at least one tube on the deployment device. In yet an additional and/or alternative non-limiting embodiment of the invention, the deployment device includes at least one marker to identify the distal end of at least one tube on the deployment device. In still yet an additional and/or alternative non-limiting embodiment of the invention, the deployment device includes at least one marker to identify the location of one or more fixtures on the deployment device. In an additional and/or alternative non-limiting embodiment of the invention, the deployment device includes at least one marker to identify the location of one or more flare portions on one or more tubes of the deployment device. In still an additional and/or alternative non-limiting embodiment of the invention, the deployment device includes at least one marker to identify the location of one or more taper portions on one or more tubes of the deployment device.

In still an additional and/or alternative non-limiting aspect of the present invention, the deployment device can include medical device securing arrangement that is designed to inhibit or prevent the medical device from undesirable movement relative to one or more tubes of the deployment device during the deployment of the medical device. As mentioned above, axial movement of the medical device on the deployment device during the deployment of the medical device can result in the improper positioning of the medical device in a body passageway. The medical device securing arrangement is designed to reduce or prevent such axial movement of the medical device during deployment of the medical device. In one non-limiting embodiment of the invention, the medical device securing arrangement includes a mechanical arrangement to releasably secure the medical device to the second tube (medical device mounting tube). Such mechanical arrangements can include, but are not limited to, latch, hook, lock, wire retainer, hook and loop fastener (e.g., Velcro, etc.), clamp, tongue and groove arrangements, etc. In one non-limiting aspect of this embodiment, a wire retainer arrangement is used to at least partially releasably secure a medical device to the second tube. The one or more wires used in the wire retainer arrangement can be a metal and/or non-metal wire. In one non-limiting design for this aspect, the one or more wires are designed to at least partially located inside the second tube. The end of the one or more wires can be secured to a release arrangement for the medical device and/or directly secure the medical device to the second tube; however, this is not required. When the end of the one or more wires directly secures the medical device to the second tube, the second tube typically includes one or more openings to enable the end of the wire to exit the interior of the second tube and engage at least a portion of the medical device; however, this is not required. The movement of the one or more wires in the second tube can be designed to cause the medical device to be at least partially released from the second tube. In another non-limiting design, the medical device securing arrangement can be integrated with the first tube (medical device deployment tube) and/or the third tube (medical device cover) in a manner that when the third tube is moved so as to be partially or fully removed from the medical device, the medical device securing arrangement releases the medical device from the second tube (medical device mounting tube). In this arrangement, the release of the medical device from the medical device securing arrangement is based on the movement of the first or second tube. For instance, in one non-limiting configuration, a wire can be secured to the first and/or third tube. One end of the wire can secures the medical device to the second tube and/or is secured to a mechanism that is used to secure the medical device to the second tube. The other end of the wire is secured to the first or third tube. When the first tube is moved to cause the third tube to axially move off the medical device, the wire can be designed to release and/or cause the release of the medical device from the second tube. This release of the medical device can be designed to occur after a certain amount of the third tube is moved off of the medical device; however, this is not required. As can be appreciated, may other arrangements can be used to release the medical device from the second tube. In an additional and/or alternative non-limiting embodiment of the invention, the medical device securing arrangement can be designed to enable a user to manually, mechanically or electronically cause the at least partial release of the medical device from the second tube.

A method of deploying a medical device such as, but not limited to, a stent in accordance with the present invention includes inserting the novel deployment device inside of a patient's body by moving the deployment device through a body passageway and to a location to be treated by the medical device. Generally, a guide catheter is inserted into the body passageway prior to inserting the novel deployment device; however, it can be appreciated that both devices can be inserted together into the body passageway. The method of deploying the medical device in a body passageway includes deploying at least a portion of a proximal end of a medical device prior to partially or fully deploying a portion of a distal end of the medical device in the body passageway. In one non-limiting embodiment, the novel deployment device includes a first tube, a second tube, and a third tube. The first tube (e.g., medical device deployment tube) has an inner diameter configured to receive an associated guide wire or other type of guiding device. The second tube (e.g., medical device mounting tube) has an inner diameter configured to receive the first tube and has an outer diameter configured to be received by an associated stent or other type of medical device. The third tube (e.g., medical device cover) has an inner diameter that receives the stent or other type of medical device. The first tube contacts and/or engages and/or is connected to the third tube. Such contact and/or engagement is generally at or adjacent a distal end of the first tube; however, this is not required. The third tube is designed to deploy a stent or other type of medical device by moving the third tube in an axial direction with respect to the second tube. The one or more tubes of the novel deployment device generally have a circular cross-sectional area; however, it can be appreciated that other shapes can be used for one or more of the tubes (e.g., oval, polygonal, etc.). The method of deployment includes moving the first tube and the third tube further in the first axial direction while retaining the second tube in position such that the stent or other type of medical device is no longer disposed between the second tube and the third tube, and thus is allowed to deploy inside a body passageway (e.g., blood vessel) of the patient. In an additional and/or alternative non-limiting embodiment, the method of deployment can include the drawing of one or more of the three the tubes through the medical device after the medical device has been at least partially deployed. In one specific method, all three tubes are drawn through the medical device after the medical device has been deployed. In still an additional and/or alternative non-limiting embodiment, the method of deployment can include the use of one or more markers on one or more of the tubes of the deployment device to 1) facilitate in informing the physician the location of the medical device and/or one or more components of the novel deployment device in a body passageway, 2) facilitate in positioning the medical device and/or one or more components of the novel deployment device in a body passageway, and/or 3) at least partially retain the medical device on and/or in position on one or more components of the novel deployment device. As can be appreciated, the one or more markers can have other or additional uses. In yet an additional and/or alternative non-limiting embodiment, the method of deployment can include the use of one or more openings in the one or more tubes of the deployment device. The one or more openings can be used to 1) facilitate in removing air from the tubes, and/or 2) allowing medicine and/or other types of fluid to be conveyed to a treatment site; however, the one or more openings can be used for other or additional reasons. In still yet an additional and/or alternative non-limiting embodiment, the method of deployment can include the use of one or more inflatable structures (e.g., angioplasty balloon, etc.) to further expand and/or properly position the medical device in the body passageway. In an additional and/or alternative non-limiting embodiment, the method of deployment can include the use of one or more low friction materials and/or lubricating coatings to facilitate in the movement of one or more components of the deployment device relative to one another. In still an additional and/or alternative non-limiting embodiment, the method of deployment can include the use of a fixture to limit or prevent movement of the medical device on one or more components of the deployment device when the medical device is being deployed in a body passageway and/or be partially retrieved back into the deployment device. In yet an additional and/or alternative non-limiting embodiment, the method of deployment can include the use of a tapered portion on one or more portions of the deployment device to facilitate in the movement of the deployment device in a body passageway. In still yet an additional and/or alternative non-limiting embodiment, the method of deployment can include the use of a flared portion on one or more portions of the deployment device to facilitate in the retrieval of a medical device back into the deployment device after the medical device has been partially deployed. In an additional and/or alternative non-limiting embodiment, the method of deployment can include the use of a mechanical securing arrangement to limit or prevent movement of the medical device on one or more components of the deployment device when the medical device is being deployed in a body passageway and/or be partially retrieved back into the deployment device.

These and other advantages will become apparent to those skilled in the art upon the reading and following of this description taken together with the accompanying drawings.

### BRIEF DESCRIPTION OF THE DRAWINGS

Reference may now be made to the drawings, which illustrate various embodiments that the invention may take in physical form and in certain parts and arrangements of parts wherein:

FIG. 1 is a side cross-sectional view of the novel deployment device.

FIG. 2 is a perspective cross-sectional view of a proximal portion of the deployment device of FIG. 1.

FIG. 3 is a close-up view, in partial cross-section, of a distal portion of the deployment device of FIG. 1.

FIG. 4 is a close-up view, in partial cross-section, of another distal portion of the deployment device of FIG. 1.

FIG. 5 is a cross-sectional view of a proximal portion of the deployment device of FIG. 1.

FIG. 6 is a close-up cross-sectional view of the proximal portion of the deployment device of FIG. 1.

FIG. 7 is a front end view of the deployment device of FIG. 1.

FIG. 8 is a cross-sectional view of a modified proximal end of the deployment device of FIG. 1.

FIG. 9 is a close-up cross-sectional view of the modified proximal end of the deployment device of FIG. 1.

FIG. 10 is a cross-sectional view of a modified proximal end of the deployment device of FIG. 1.

FIG. 11 is a close-up cross-sectional view of the modified proximal end of the deployment device of FIG. 10.

FIGS. 12-15 are views of the deployment device of FIG. 1 inserted into a patient's body passageway showing a novel deployment method of a medical device inside of the body passageway.

### DETAILED DESCRIPTION OF THE INVENTION

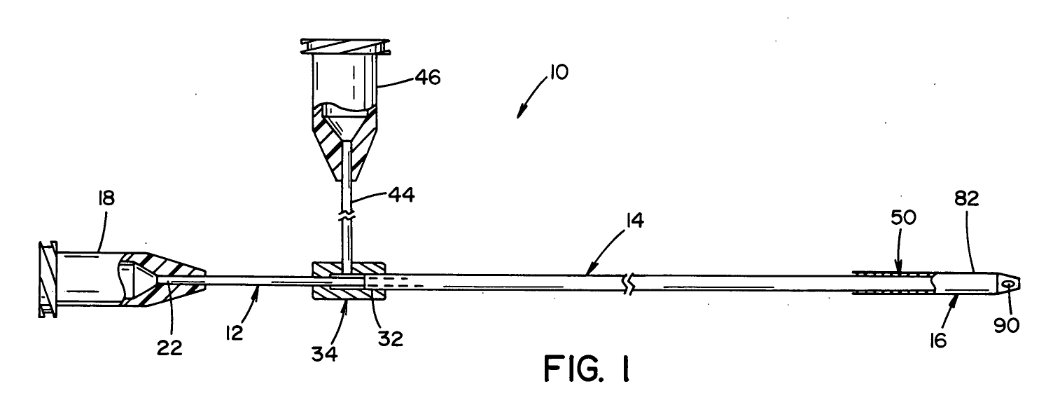

Referring now to the drawings wherein the showings are for the purpose of illustrating embodiments of the invention only and not for the purpose of limiting the same, FIG. 1 illustrates a deployment device 10 that generally includes a first tube 12, a second tube 14, and a third tube 16. The deployment device 10 is useful for delivering medical devices, for example stents, inside of a patient's body passageway. The tubes 12, 14 and 16 are designed to travel at least partially through a particular patient's body passageway and therefore are appropriately dimensioned. Tubes 12 and 14 are generally made from a flexible, biocompatible material. Tube 16 can also be formed of a flexible material; however, the material can be rigid. Non-limiting examples of materials that can be used to form one or more of the tubes include, but are not limited to, plastic, metal, fiber reinforced materials, etc. The materials used to form the different tubes can be the same or different. The material used to form the one or more tubes can be the same throughout the longitudinal length of the tube, or can vary along the longitudinal length of the tube. The exterior and/or interior cross-sectional shape, and/or thickness of one or more tubes can be the same or different along the longitudinal length on the one or more tubes. A lubricious coating and/or low friction surface material can be applied to and/or be used on one or more of the tubes to facilitate in the movement of the tubes relative to one another; however, this is not required. In the depicted embodiment, the tubes 12, 14 and 16 are generally circular in interior and exterior cross-section; however, it will be appreciated that the tubes can take other configurations.

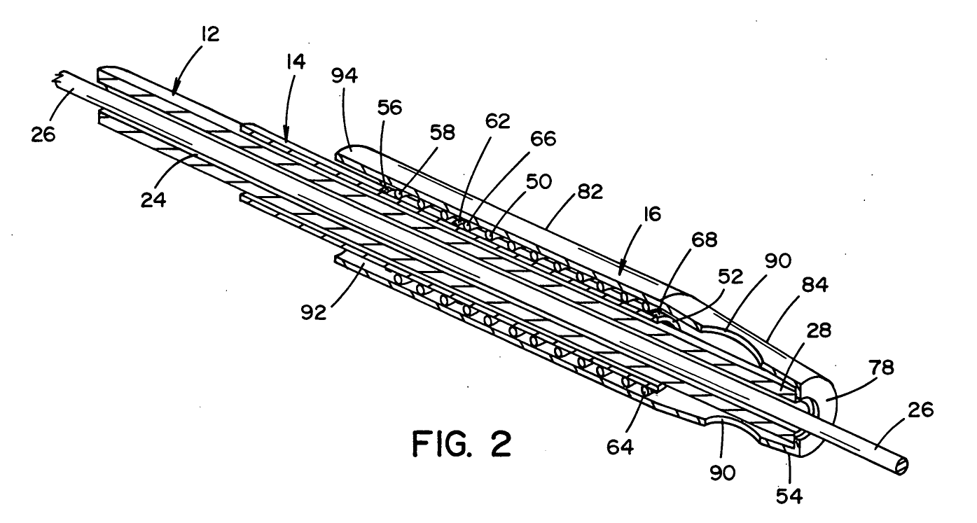

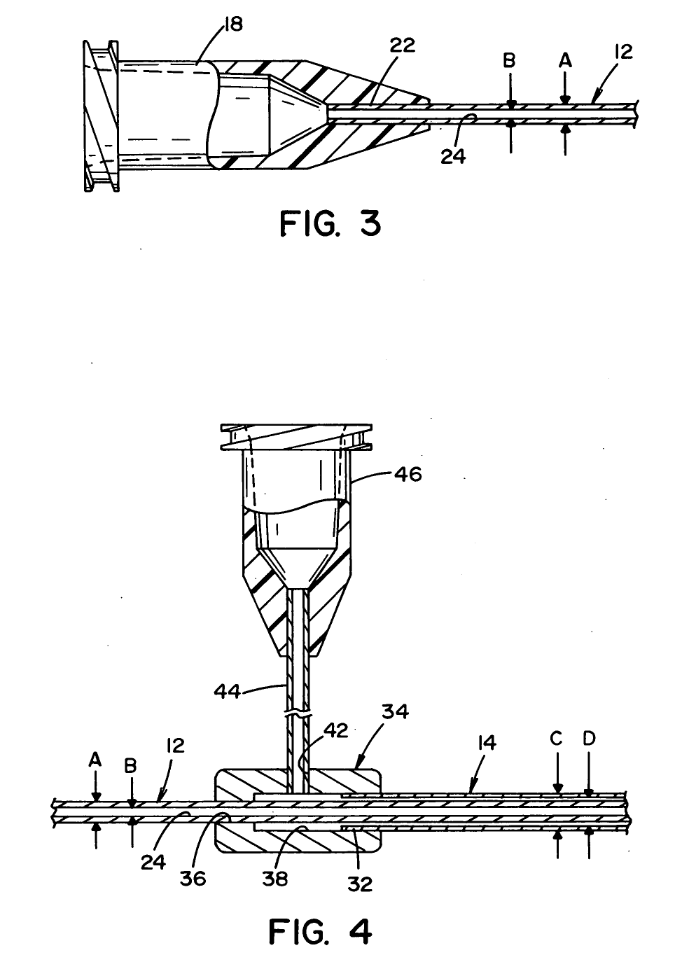

A proximal end of the deployment device 10 is depicted in FIG. 2. The first tube 12 can be referred to as a deployment tube. The second tube 14 can be referred to as a mounting tube. The third tube 16 can be referred to as a cover. With reference to FIGS. 1 and 3, the deployment tube 12 is designed to attach to a first Luer fitting 18 at or near the distal end 22 of the deployment tube; however, this is not required. Luer fittings are known in the art, thus will not be further described herein. With reference to FIGS. 2 and 3, the deployment tube 12 has an outer diameter “A” or outer cross-sectional area, and also includes an internal passage 24 having an inner diameter B or inner cross-sectional area that is dimensioned to receive a guide wire 26. The length of the deployment tube will vary depending on the type of procedure to be used. Generally the length of the deployment tube when used to deploy a medical device, such as a stent near the heart of an average adult, is about 90-180 cm; however, other lengths can be used. The inner diameter and outer diameter of the deployment tube, when used with a 0.014 inch diameter guide wire is used, is about 0.015-0.018 inch and 0.032-0.036 inch respectively; however, other inner and outer diameters of the deployment tube can be used. The guide wire is generally used to guide the deployment device 10 at least partially through a patient's body passageway, e.g., a blood vessel. Fluid, for example, drugs, saline, air and the like can be introduced into and/or flushed from the internal passage 24 of the deployment tube 12 via the Luer fitting, which is in communication with the internal passage. Commonly, the Luer fitting is used to remove air from internal passageway 24; however, this is not required.

With reference now to FIG. 2, proximal end 28 of the deployment tube 12 contacts the proximal end of cover 16. In the depicted embodiment, the deployment tube 12 is integrally formed with or bonded to the cover 16; however, it can be appreciated that deployment tube 12 can be connected to cover 16 in other or additional ways. As can also be appreciated, deployment tube 12 can be designed to contact and/or be attached to the cover 16 at locations that are spaced from proximal end 28 of the deployment tube 12 and/or the proximal end of cover 16.

The deployment tube 12 has an outer diameter “A” or outer cross-sectional area. With reference to FIG. 4, the outer diameter or outer cross-sectional area of the deployment tube 12 measures less than an inner diameter “D” or inner cross-sectional area of the mounting tube 14 so that at least a portion of the deployment tube 12 is received inside and can be moved within the mounting tube 14. A distal end 32 of mounting tube 14 can be designed to attach to a connector 34. For instance, if the outer diameter “B” of the deployment tube was 0.034 inch, the inner diameter “D” would generally be about 0.035-0.038 inch and the outer diameter “C” would be about 0.038-0.042 inch; however, it will be appreciated that other inner and/or outer diameters of the mounting tube can be used. The length of the mounting tube is typically less than the length of the deployment tube. The connector 34 includes a through bore 36 that receives the deployment tube 12 such that the deployment tube extends completely through the connector; however, this is not required. A counterbore 38 is coaxial with the through bore 36. The counterbore 38 is slightly larger than the through bore 36 and is dimensioned to receive mounting tube 14. The connector 34 also includes a transverse passage 42 that is in communication with the counterbore 38. The transverse passage 42 receives a fourth tube 44 that is attached to a second Luer fitting 46. The inner diameter “D” or inner cross-sectional area of mounting tube 14 and the outer diameter “A” or outer cross-sectional area of deployment tube 12 are configured such that a small clearance is provided between the deployment tube and the mounting tube. Fluid such as, but not limited to, drugs, saline, and/or air can be delivered into and/or removed from the deployment device via the second Luer fitting 46 and the fourth tube 44 through the connector 34 and into the clearance between deployment tube 12 and mounting tube 14. As can be appreciated, the cross-sectional shape of the mounting tube and deployment tube is typically circular; however, other shapes can be used (e.g., oval, etc.). The size of the cross-sectional area of the mounting tube and deployment tube is generally constant along the longitudinal length of such tubes; however, this is not required.

As illustrated in FIG. 4, mounting tube 14 has an outer diameter “C” or outer cross-sectional area that is configured such that the mounting tube is received inside a medical device such as, but not limited to, a stent 50. As illustrated in FIGS. 2, 5 and 6, stent 50 is not in a fully expanded state. Stent 50 in the depicted embodiment can be a known type of stent that is made of a material such as, but not limited to, stainless steel, cobalt chromium, magnesium, and/or nitinol. The stent can be designed to at least partially self expand; however, this is not required. The stent can have a variety of shapes and/or sizes. Non-limiting examples of stents that can be used include stents disclosed and/or cited in the prior art in U.S. Pat. Nos. 4,994,071; 5,456,712; 5,466,242; 5,607,144; 6,206,916; 6,293,964; 6,436,133; and US Patent Publication Nos. 2005/0171596; 2005/0165476; 2005/0159802; 2004/0181277; 2003/0040790; 2002/0099438, all of which are incorporated herein by reference.

As illustrated in FIGS. 2 and 6, stent 50 is disposed between mounting tube 14 and cover 16. Stent 50 is positioned around a proximal portion of mounting tube 14. A proximal end 52 of mounting tube 14 is axially spaced from the proximal end 28 from the deployment tube 12 and proximal end 54 of cover 16; however, this is not required. The stent can be at least partially secured to mounting tube 16 by a friction engagement, an adhesive, etc.; however, this is not required. In the depicted embodiment, mounting tube 14 includes one or more markers (e.g., radiopaque markers or other known markers used in arterial procedures) to indicate the position of the stent on mounting tube 14 and/or the position of the stent in a body passageway. As can be appreciated, other or additional markers can be used on the cover, deployment tube 12, stent 50, and/or mounting tube 14 to provide position information regarding the stent and/or one or more components of deployment device 10. The markers can be made of a variety of materials (e.g., metal, polymer, etc.). As illustrated in FIG. 2, a first marker 56 is disposed on mounting tube 14 adjacent a proximal end 58 of the stent 50. A second marker 62 is axially spaced from the first marker 56 toward a distal end 64 of the stent 50 and is disposed on the mounting tube 14 adjacent a transition portion 66 of the stent, which will be described in more detail below. A third marker 68 is axially spaced from the second marker 62 and is disposed on the mounting tube 14 adjacent the distal end 64 of the stent. The markers 56, 62, 68 can include a wall that extends radially from the mounting tube 14 toward the cover 16; however, this is not required. As can be appreciated, one or more markers can also or alternatively be directly located on the stent and/or on the first and/or third tube. The one or more markers, in addition to providing a locating function for the stent 50, can also aid in mounting the stent 50 between the cover 16 and the mounting tube 14; however, this is not required. In one non-limiting design, marker 56 and/or 68 can be used to at least partially prohibit axial movement of the stent 50 along mounting tube 14. In addition or alternatively, a fixture or stopper 70 as illustrated in FIG. 9 can be used to inhibit or prevent axial movement of stent 50 along mounting tube 14. Fixture 70 is shown to extend to the inner surface of cover 16; however, this is not required. Fixture 70 can be a separate component from mounting tube 14 (e.g., plastic ring, metal ring, etc.) or be integrally formed on the proximal end portion of the mounting tube. The fixture can include a marker material; however, this is not required. The one or more markers and/or fixtures can be used to limit or prevent axial movement of the stent on the mounting tube when 1) the stent is mounted between the cover 16 and the mounting tube 14, 2) when the stent is being inserted into a body passageway, 3) when the stent is deployed in the body passageway, and/or 4) when the stent is retrieved after partial deployment of the stent.

As illustrated in FIG. 6, cover 16 is disposed along a proximal portion of the deployment device 10. In the depicted embodiment, cover 16 includes a proximal end wall 78 that is generally normal to a longitudinal axis 80 of the deployment device 10. The proximal end 28 of the deployment tube 12 contacts the proximal end wall 78 of cover 16. As mentioned above, the proximal end 28 of deployment tube 12 engages and/or is connected to the proximal end wall 78 so that movement of the deployment tube 12 in at least one axial direction (insertion and/or retraction) results in movement of the cover 16. As mentioned above, cover 16 can be connected to deployment tube 12 in other or additional locations. The wall thickness of deployment tube 12 is generally thicker than cover 16; however, this is not required. In one non-limiting configuration, the deployment tube 12 is about 1.5-5 times the wall thickness of cover 16; however, other thickness ratios can be used. Deployment tube 12 is designed to endure the normal compressive forces required to move the cover 16 in an axial direction during the deployment of the stent.

In the depicted embodiment, the cover 16 is shaped to include a generally cylindrical portion 82 that surrounds the stent 50 and a tapered portion 84 that tapers downwardly (distal to proximal) along longitudinal axis 80. The generally cylindrical portion has an inner diameter that is sufficient to enable a stent to be placed in space 90 between the inner surface of cover 16 and the outer surface of mounting tube 14. In one non-limiting design, when the outer diameter “C” of the mounting tube is about 0.036 inch, the inner diameter of the cover is about 0.046-0.05 inch and the outer diameter of the cylindrical portion of the cover is about 0.05-0.054; however, it will be appreciated that other inner and/or outer diameter sizes for the cover can be used. The taper can be a constant taper as illustrated in FIG. 6, or can be a non-constant taper. As can be appreciated, cover 16 can have other configurations. As can also be appreciated, the tapered portion of cover 16 is not required. When the tapered portion is used, the tapered portion facilitates in the movement of the tubes and stent in a body passageway. As more clearly seen in FIG. 2, the cylindrical section 82 of the cover 16 is radially spaced from the mounting tube 14. As also illustrated in FIG. 2, the length of cover 16 is greater than stent 50; however, this is not required. Generally, the length of cover 16 is about 1-3 times the length of stent 50, and typically about 1.1-2 times the length of the stent; however, it will be appreciated that other length ratios can be used. A space 92 is defined between a distal end 94 of cover 16 and the mounting tube 14. Stent 50 is positioned in space 92 between the inner surface of cover 16 and the outer surface of mounting tube 14.

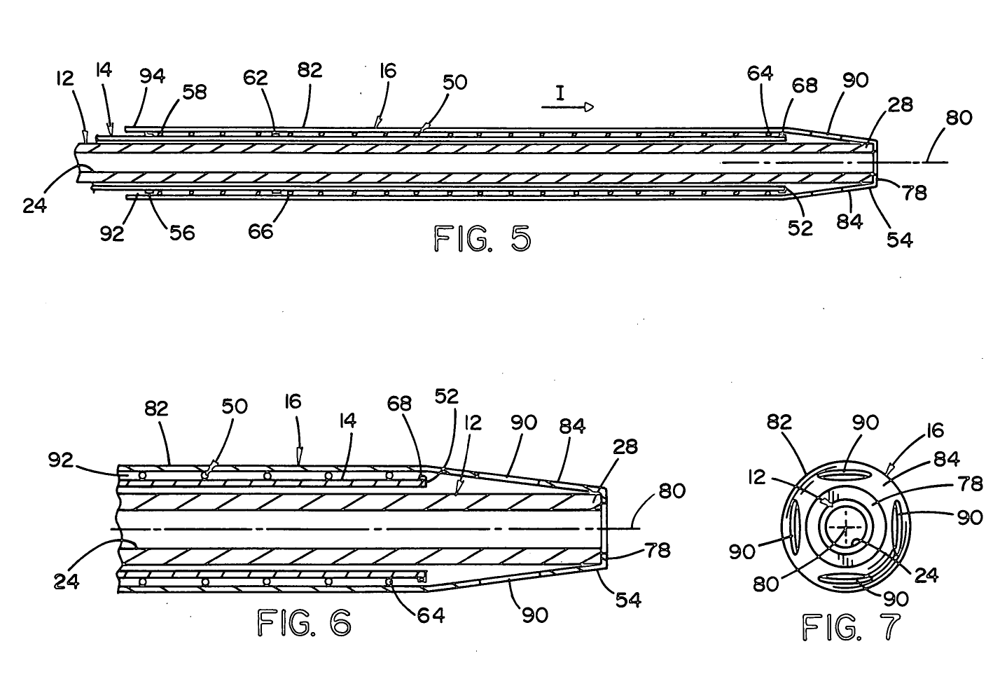

With reference to FIGS. 2 and 7, cover 16 can include fluid openings 90. In the depicted embodiment, four elliptical fluid openings 90 are provided spaced about 90° apart from one another; however, other numbers of openings and/or angles can be used. As can also be appreciated, one or more fluid openings can be positioned at the end of the cover and/or in other or additional locations. Although not shown, deployment tube 12 and/or mounting tube 14 can also include one or more fluid openings. The fluid openings are generally used to allow for fluid, e.g., blood, to enter into the fluid openings and pass between two or more of the components of the deployment device 10 as the deployment device is moved in an insertion direction I (FIG. 5), which is typically parallel to the longitudinal axis 80 of the deployment device. The fluid openings are generally used to flush air from the deployment device; however, one or more of the fluid openings can have other or additional uses.

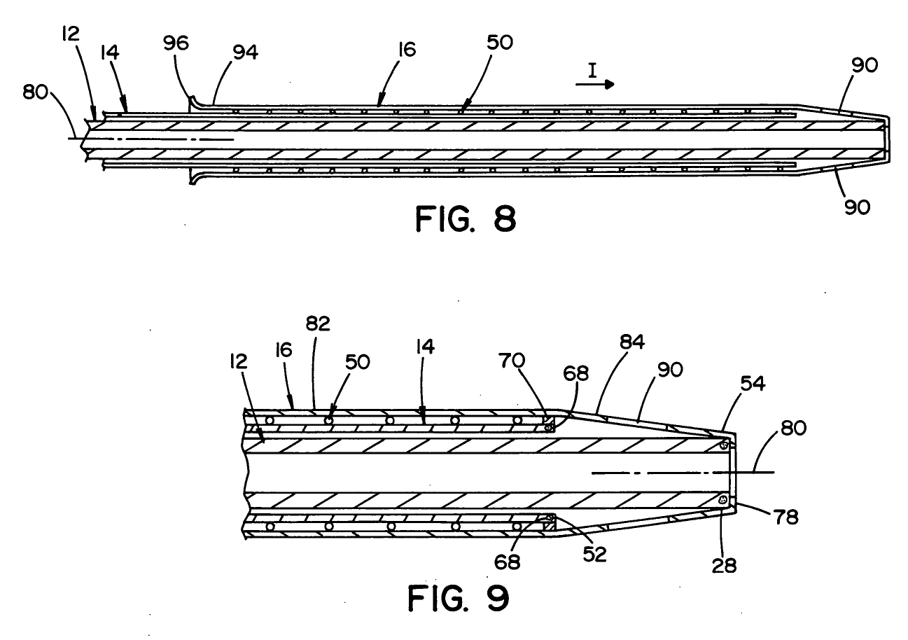

Referring now to FIG. 8, the distal portion 94 of cover 14 can include a flare portion 96. The flare portion can be used to retrieve a partially deployed stent 50 back between cover 14 and mounting tube 16. Typically, the flare portion is less than 90° relative to longitudinal axis 80.

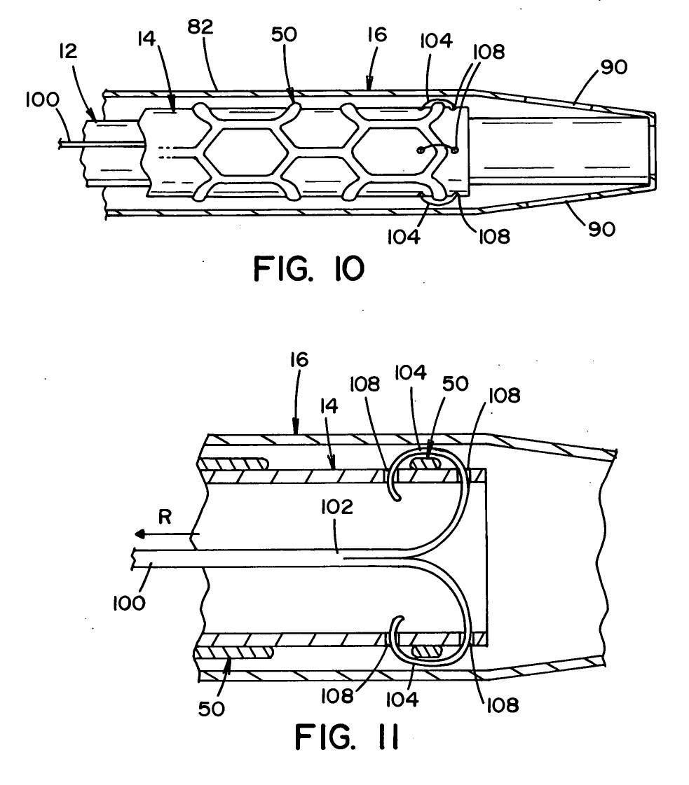

Referring now to FIGS. 10 and 11, a mechanical securing arrangement is used to secure the stent 50 to mounting tube 14. The mechanical securing arrangement, like fixture 70, is designed to limit the axial movement of the stent on the mounting tube during the deployment of the stent. The mechanical arrangement includes a release wire 100 that is designed to mechanically release the stent from the mounting tube. The release wire typically includes a flexible material; however, this is not required. Materials that can be used for the release wire include metal, synthetic materials (nylon, Kevlar, etc.), fiber reinforced materials, etc. As mention above, the stent can be at least partially secured to the mounting tube by an adhesive; however, this is not required. As illustrated in FIG. 11, the mounting tube includes a plurality of openings 108. FIG. 11 does not show deployment tube 12 so as to simply the description of this embodiment. These openings are designed to enable an end portion 102 of the release wire to be inserted therethrough. The end portion 102 of the release wire is split into a plurality of strands 104; however, this is not required. As illustrated in FIG. 11, the strands 104 pass through opening 108 and loop over a portion of stent 50 thereby securing the stent to the mounting tube. The stent can be released from the mounting tube by withdrawing the release wire 100 in direction “R” as illustrated in FIG. 11. By pulling the release wire in direction “R” the strands 104 are drawn through openings 108 and into the interior of the mounting tube. The embodiment illustrated in FIGS. 10 and 11 show release wire 100 as being positioned in mounting tube 14. As can be appreciated, the release wire could be positioned in deployment tube 12 or in some other tube or arrangement.

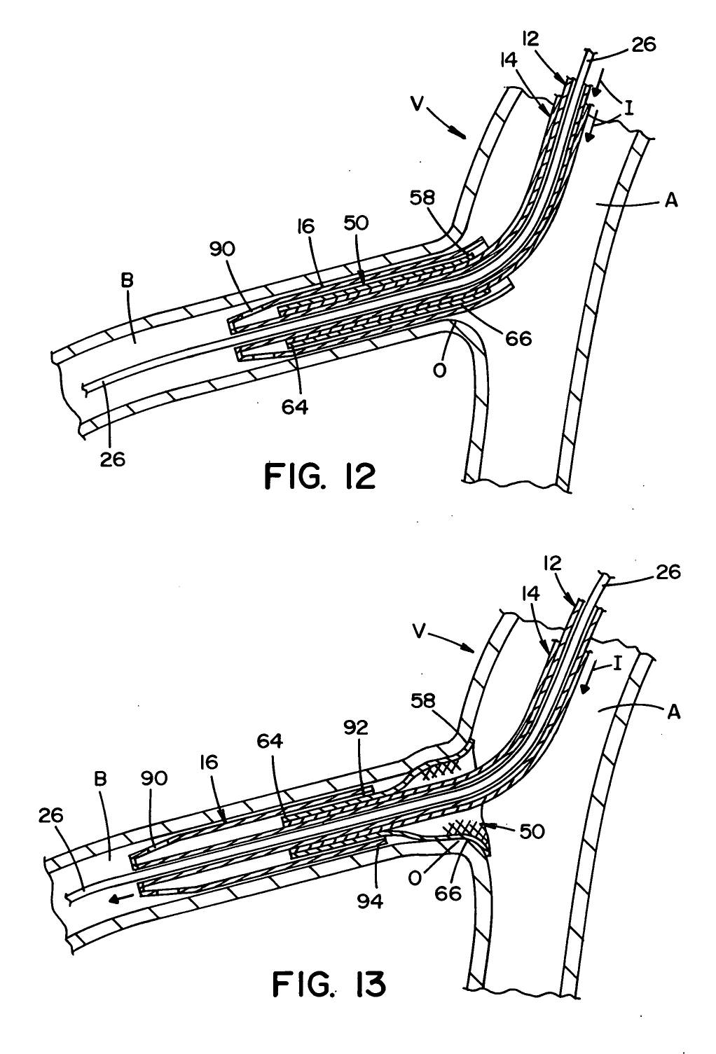

The deployment device 10 can be used to deliver a medical device such as a stent inside a patient's blood vessel; however, the deployment device can be used to deploy other types of medical devices in a blood vessel or other types of body passageways. FIGS. 12-15 depict one non-limiting method for deploying a stent inside a patient's blood vessel V. The general teachings of this method can be applied to delivering a medical device inside other body passageways. FIGS. 12-15 depict a first artery A and a second artery B that branches from the first artery. Plaque, not shown, tends to build up at the intersection of the two arteries (e.g., the ostial of the left main coronary artery, the ostial of the right coronary artery, the ostial of the innominate artery, the ostial of the left common carotid artery, the ostial of the subclavian artery, the ostial of vertebral arteries, the ostial of renal arteries, the ostial of the hepatic artery, and the ostial of mesenteric arteries, etc.). As can be appreciated, plaque and/or other problems in an artery are not limited to the ostial region of the artery. As such, the deployment device of the present invention can be used to deploy a stent in all regions of a blood vessel. When a stent is to be deployed in the ostial region of the artery, a specially designed stent is commonly used for such applications. Non-limiting examples of such stents are disclosed in U.S. Pat. Nos. 4,994,071; 5,456,712; 5,466,242; 5,607,144 and 6,293,964, all of which are incorporated herein by reference. Another non-limiting stent design for such region of the artery is also disclosed in Patent Application Ser. No. 60/627,421, which is also incorporated herein by reference. One non-limiting general design of the stent disclosed in Patent Application Ser. No. 60/627,421 is illustrated in FIGS. 12-15. The stent is useful in opening a blockage that occurs at the ostium of the second artery B. The stent in an expanded state is a trumpet-shaped stent. The non-flaring end of the stent is delivered into the second artery B and the flaring end of the stent contacts the walls of the first artery A.

When implanting a flaring stent 50 inside the patient's blood vessel by use of the novel deployment device, a catheter (not shown) is delivered through the blood vessel(s), e.g., artery A, of the patient such that a distal end of the catheter terminates at or near the blockage or occlusion in the blood vessel which in the FIGS. 12-15 would be at the ostium 0 of the second artery B. The guide wire 26 is then inserted into the catheter up to the distal end of the catheter and past the occlusion. The deployment device 10 is then inserted into the catheter such that the deployment tube 12 receives the guide wire 26 to guide the deployment device up to or near the occlusion or blockage in the blood vessel of the patient. The proximal portion of the deployment device 10 is then inserted past the occlusion so that the stent 50 is properly located so that when the stent is deployed, it opens the occlusion.

To deploy the stent 50, the deployment tube 12 is moved in a further insertion direction, as indicated by arrow I in FIGS. 12 and 13, past the occlusion. While the deployment tube is being moved, mounting tube 14 is restrained from moving in the insertion direction. The deployment tube 12 moves the cover 16 in the general direction of arrow I. As seen in FIG. 13, the mounting tube 14 is retained such that the stent 50 remains in position as cover 14 exposes the proximal end of the stent. The radiopaque marker(s) 56, 62 and 68 (FIG. 2) are used to indicate the position of stent 50 during deployment. When mounting tube 14 includes a fixture 70 or one or more elevated markers, the fixture and/or elevated markers inhibit or prevent axial movement of the stent as the cover 16 is moved off the stent.

Using this delivery method, the proximal end 58 of the stent 50 is delivered first and a distal end 64 of the stent is delivered last. By delivering the proximal end 58 of the stent 50 first, followed by the distal end, the stent does not have a tendency to “jump” past the occlusion. This is especially a useful result when deploying the stent in a certain region such as the ostium of the artery. When positioning the stent 50 prior to deployment, the markers 58, 62 and 68 can be used to indicate the position of the stent inside the artery. The second marker 62 can be positioned in the ostium O of the second artery B so that the transition 66 of the stent (the general area and/or point at which the flared end begins) is appropriately positioned to allow the proximal, i.e., flared, end 58 of the stent to expand against the walls of the first artery A. In this arrangement, the physician is able to observe the location of the stent in the artery and to determine where the flared portion of the stent should be positioned. If the physician determines that after the flared portion of the stent is deployed that the flared portion is space incorrectly at the ostium. The physician is able to insert or retract the partially deployed stent in the artery until the flared portion is property positioned as indicated by the markers and/or other positioning aids. Thereafter, the remainder of the stent can be deployed to fully seat the stent in the artery. Such an insertion method lessens the likelihood of the flared end of the stent 50 extending into the first artery A and away from the ostium O of the second artery B, which is undesirable.

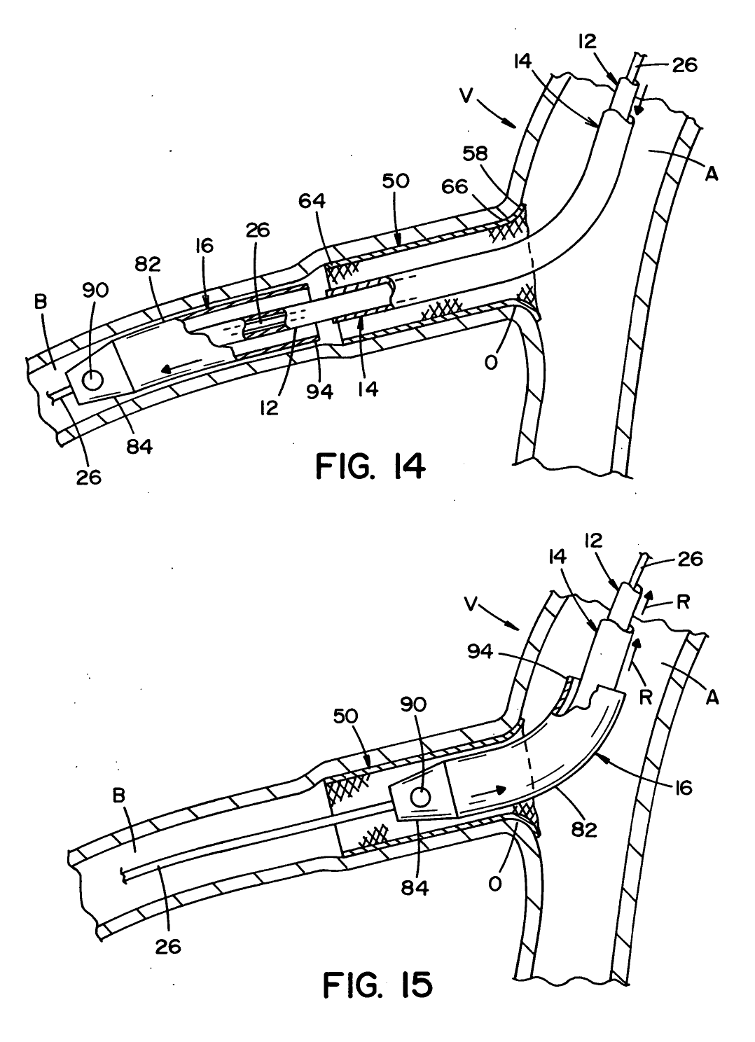

With reference to FIGS. 14 and 15, when cover 14 is removed from the stent, the stent is fully deployed in the artery. The deployed stent 50 expands such that the inner diameter of the stent 50 is larger than the outer diameter of the cover 16. The deployment device 10 is then pulled in a retracting direction as depicted by arrow R in FIG. 15, such that the cover is pulled through the interior region of the stent. The retracting direction is generally parallel to a longitudinal axis of the deployment device 10. The guide wire 26 and the catheter are thereafter removed.

The deployment device 10 can be used in conjunction with other procedures. For example, after the deployment device 10 has deployed stent 50 in the artery, the deployment device can be used to deploy another stent in artery A so as to T-stent the two arteries. In additional or alternatively, once stent 50 has been deployed as shown in FIG. 15, an angioplasty balloon can be inserted inside of the stent 50 inside of the second artery B. The balloon can be used to open the stent 50 and to press any blockage against the vessel wall of the second artery B. Another angioplasty balloon can be inserted into the first artery A adjacent the flared end 58 of the stent 50; however, this is not required. The second balloon, when used, is inflated to press the flared end 58 of the stent against the vessel wall of the first artery A prior to the first balloon being inflated.

A deployment device and a method for inserting a medical device into a body passageway of a patient has been described in such a manner so that a person skilled in the art can make and use the aforementioned device and practice the aforementioned method. Modifications and alterations will occur to those upon reading and understanding the detailed description that has been provided above. The invention is not limited to only the depicted embodiments and the two described methods. Instead, the invention is broadly defined by the appended claims and the equivalents thereof.

We claim:

1. A deployment device for deploying a medical device in a body passageway, said deployment device designed to deploy at least a portion of a proximal end of the medical device before fully deploying a distal end of the medical device.

2. The deployment device as defined in claim 1, including a medical device mounting structure that at least partially supports the medical device, a medical device cover that at least partially covers a portion of the medical device, and a deployment structure, said medical device cover and said deployment structure axially movable relative to a longitudinal axis of said medical mounting structure.

3. The deployment device as defined in claim 2, wherein said medical device cover is designed to engage said deployment structure.

4. The deployment device as defined in claim 2, wherein said medical device cover is connected to said deployment structure.

5. The deployment device as defined in claim 2, wherein said medical device mounting structure including a cavity at least partially along a longitudinal axis of said medical device mounting structure, said deployment structure designed to be at least partially telescopically inserted into said cavity of said medical device mounting structure, said cover including a cavity at least partially along a longitudinal axis of said cover, said medical device mounting structure designed to be at least partially telescopically inserted into said cavity of said cover.

6. The deployment device as defined in claim 3, wherein said medical device mounting structure including a cavity at least partially along a longitudinal axis of said medical device mounting structure, said deployment structure designed to be at least partially telescopically inserted into said cavity of said medical device mounting structure, said cover including a cavity at least partially along a longitudinal axis of said cover, said medical device mounting structure designed to be at least partially telescopically inserted into said cavity of said cover.

7. The deployment device as defined in claim 4, wherein said medical device mounting structure including a cavity at least partially along a longitudinal axis of said medical device mounting structure, said deployment structure designed to be at least partially telescopically inserted into said cavity of said medical device mounting structure, said cover including a cavity at least partially along a longitudinal axis of said cover, said medical device mounting structure designed to be at least partially telescopically inserted into said cavity of said cover.

8. The deployment device as defined in claim 2, including at least one marker on a structure selected from the group consisting of said medical device mounting structure, said deployment structure, said medical device cover, or combinations thereof.

9. The deployment device as defined in claim 5, including at least one marker on a structure selected from the group consisting of said medical device mounting structure, said deployment structure, said medical device cover, or combinations thereof.

10. The deployment device as defined in claim 6, including at least one marker on a structure selected from the group consisting of said medical device mounting structure, said deployment structure, said medical device cover, or combinations thereof.

11. The deployment device as defined in claim 7, including at least one marker on a structure selected from the group consisting of said medical device mounting structure, said deployment structure, said medical device cover, or combinations thereof.

12. The deployment device as defined in claim 2, including a fixture on said medical device mounting structure, said fixture designed to at least partially inhibit axial movement of said medical device on said medical device mounting structure as said cover is removed from said medical device.

13. The deployment device as defined in claim 5, including a fixture on said medical device mounting structure, said fixture designed to at least partially inhibit axial movement of said medical device on said medical device mounting structure as said cover is removed from said medical device.

14. The deployment device as defined in claim 6, including a fixture on said medical device mounting structure, said fixture designed to at least partially inhibit axial movement of said medical device on said medical device mounting structure as said cover is removed from said medical device.

15. The deployment device as defined in claim 7, including a fixture on said medical device mounting structure, said fixture designed to at least partially inhibit axial movement of said medical device on said medical device mounting structure as said cover is removed from said medical device.

16. The deployment device as defined in claim 9, including a fixture on said medical device mounting structure, said fixture designed to at least partially inhibit axial movement of said medical device on said medical device mounting structure as said cover is removed from said medical device.

17. The deployment device as defined in claim 12, wherein said fixture has a height that is at least 25% of a maximum space between an outer surface of said medical device mounting structure and an inner surface of said cover when that is formed when said medical device mounting structure is at least partially telescopically inserted in said cavity of said cover.

18. The deployment device as defined in claim 16, wherein said fixture has a height that is at least 25% of a maximum space between an outer surface of said medical device mounting structure and an inner surface of said cover when that is formed when said medical device mounting structure is at least partially telescopically inserted in said cavity of said cover.

19. The deployment device as defined in claim 2, including a fluid opening on a structure selected from the group consisting of said medical device mounting structure, said deployment structure, said medical device cover, or combinations thereof.

20. The deployment device as defined in claim 5, including a fluid opening on a structure selected from the group consisting of said medical device mounting structure, said deployment structure, said medical device cover, or combinations thereof.

21. The deployment device as defined in claim 18, including a fluid opening on a structure selected from the group consisting of said medical device mounting structure, said deployment structure, said medical device cover, or combinations thereof.

22. The deployment device as defined in claim 2, including a lubricant to facilitate in movement of at least two structures relative to one another, said structures selected from the group consisting of said medical device mounting structure, said deployment structure, said medical device cover, or combinations thereof.

23. The deployment device as defined in claim 5, including a lubricant to facilitate in movement of at least two structures relative to one another, said structures selected from the group consisting of said medical device mounting structure, said deployment structure, said medical device cover, or combinations thereof.

24. The deployment device as defined in claim 21, including a lubricant to facilitate in movement of at least two structures relative to one another, said structures selected from the group consisting of said medical device mounting structure, said deployment structure, said medical device cover, or combinations thereof.

25. The deployment device as defined in claim 1, wherein said medical device is a stent.

26. The deployment device as defined in claim 2, wherein said medical device is a stent.

27. The deployment device as defined in claim 5, wherein said medical device is a stent.

28. The deployment device as defined in claim 24, wherein said medical device is a stent.

29. The deployment device as defined in claim 2, wherein said cover includes a tapered portion, a flare portion, or combinations thereof.

30. The deployment device as defined in claim 5, wherein said cover includes a tapered portion, a flare portion, or combinations thereof.

31. The deployment device as defined in claim 28, wherein said cover includes a tapered portion, a flare portion, or combinations thereof.

32. The deployment device as defined in claim 2, including a release arrangement designed to releasably secure said medical device to medical device mounting structure, said release mechanism at least partially positioned in said medical device mounting structure, said deployment structure, or combinations thereof.

33. The deployment device as defined in claim 5, including a release arrangement designed to releasably secure said medical device to medical device mounting structure, said release mechanism at least partially positioned in said medical device mounting structure, said deployment structure, or combinations thereof.

34. The deployment device as defined in claim 31, including a release arrangement designed to releasably secure said medical device to medical device mounting structure, said release mechanism at least partially positioned in said medical device mounting structure, said deployment structure, or combinations thereof.

35. A method for deploying a medical device inside a passageway of a mammalian host, the method comprising:
inserting a deployment device into a passageway of a host, said deployment device at least partially supporting said medical device that is configured to open in the passageway of the host;
deploying a proximal end of the medical device; and
deploying a distal end of the medical device after deploying at least a portion of the proximal end of the medical device.

36. The method of claim 35, wherein the medical device comprises a stent.

37. A medical device deployment instrument comprising:
a deployment structure;
a medical device mounting structure adapted to at least partially telescopically receive said deployment structure in an internal passageway in said medical device mounting structure; and,
a medical device cover adapted to at least partially cover a medical device that is at least partially positioned on said medical device mounting structure, said medical device cover at least partially connected to said deployment structure.
